(19) United States
(12) Patent Application Publication
(10) Pub. No.: US 2004/0082511 A1
(43) Pub. Date: Apr. 29, 2004

(54) Drug composition for the promotion of tissue regeneration

(76) Inventors: Georg Watzek, Vienna (AT); Reinhard Gruber, Baden (AT); Johann Eibl, Vienna (AT)

Correspondence Address: BAKER & BOTTS, 30 ROCKEFELLER PLAZA, NEW YORK, NY, 10112

(21) Appl. No.: 10/621,894

(22) Filed: Jul. 17, 2003

Related U.S. Application Data
(63) Continuation of application No. PCT/AT02/00018, filed on Jan. 17, 2002.

(30) Foreign Application Priority Data
Jan. 18, 2001 (AT) A 89/2001

(51) Int. Cl. A61K 38/39; A61K 38/36

(52) U.S. Cl. 514/12

(57) ABSTRACT

The invention relates to a drug composition to be applied topically for promoting the regeneration of tissue, characterized in that it contains microparticles from blood cells and/or tissues which have been purified by differential centrifugation, filtration or affinity chromatography, it has been subjected to a procedure for virus inactivation and/or virus depletion, it has been prepared under sterile conditions, and it is provided in freeze-dried or deep-frozen state.

The present invention relates to a drug composition for promoting the regeneration of tissue, in particular bone tissue.

If stimulated appropriately, eukaryotic cells are able to release parts of their plasma membrane into the extracellular space. Those cell fragments contain cytoplasmic moeties and are referred to as microparticles. The formation of such microparticles could be verified in monocytes, lymphocytes, endothelial cells, granulocytes and thrombocytes. In the case of thrombocytes, stimulation with collagen thrombin, Ca²⁺-ionophore A23187, and protein C5b-9 of the complement system results in exocytosis of such cellular elements (Tans G., Blood 1991; Sims P J., J Biol Chem 1988). In addition to the above-mentioned substances, which cause a modification of the intracellular calcium concentration, the formation of thrombocytic microparticles has been ascribed to protein phosphorylations, the translocation of phospholipids, changes in the cytoskeleton, and shear forces.

The microparticles of thrombocytes exhibit properties which may lead to an acceleration as well as to a slowdown of blood coagulation. By virtue of the high-affinity binding capacities of coagulation factor VIII, which is a cofactor of the tenase enzyme complex, and factor Va, which forms the prothrombinase complex with factor Xa, the microparticles are vested with a coagulation-promoting function. The binding of the factors to the surface of the microparticles is accomplished by phosphatidyl serine, a phospholipid of the cell membrane. On the other hand, the accumulation of ,,protein S” leads to an inactivation of coagulation factors Va and VIII as well as to a binding of protein C and activated protein C, resulting in an anticoagulative property of the microparticles (Tans G. Blood 1991). Compared to activated thrombocytes, the microparticles exhibit a larger number of binding sites for coagulation factors IXa (Hoffman, M., Thrombin Haemost 1992) and Va (Sims P J., JBC 1988). Furthermore, the glycoproteins GP IIb/IIIa, Ib, IaIIa and P-selectin on the cell surface render the binding of the microparticles to vascular endothelia possible (George J N JCI 1986; Gawaz M, Aterioscler Thromb Vasc Biol 1996). In addition to the activation of endothelial cells, the activation of monocytes and thrombocytes by microparticles has been demonstrated (Barry O P, Thromb Haemat 1999).

Increased concentrations of microparticles in the bloodstream have been observed in diseases associated with an activation of thrombocytes.

One of the most important functions of the immune system consists in directing leukocytes to the site of infection and the damaged tissue. In doing so, the leukocytes roll along the endothelial wall and are caused to immigrate into the wound infection area by integrins and selectins. Microparticles promote the accumulation of the ,,rolling” leukocytes by P-selectin and hence may contribute to increased haemostasis and inflammatory reactions (Forlow B. Blood 2000). The increased binding of monocytes to endothelia by microparticles has also been shown (Barry O P, JCI 1997). Another group has demonstrated that microparticles from thrombocytes result in increased proliferation of smooth muscle cells from vessels (Weber A A, Thromb Res 2000).

Thrombocytes are the smallest blood components in the human organism and may react to chemical and physical stimuli. In case of a vascular injury, thrombocytes are caused to aggregate on uncovered endothelial surfaces and to secrete a great number of biologically active substances. Among those are platelet-derived growth factor (PDGF), transforming growth factor-β(TGF-β), epidermal growth factor (EGF), metabolites of arachidonic acid such as prostaglandin D₂/E₂ and thromboxane A₂, and also microparticles.

It maybe assumed that the release of microparticles promotes the formation of the fibrin clot and the immigration of inflammatory cells into the wound area. The neutrophil granulocytes and macrophages, on their part, secrete growth factors, which, in tun, direct leukocytes, fibroblasts, and endothelial cells to immigrate into the fibrin clot. Macrophages break down destroyed tissue and promote the proliferation and synthesis of collagen type I, thereby initiating the formation of granulation tissue. Said tissue is a fibrous connective tissue that replaces the original tissue. In parallel, vessels sprout into the wound area, and the wound area is epithelized. Wound healing is completed by the formation of a cell-poor permanent scar tissue that is rich in collagen, a process that may take weeks, if not months.

Since a scar tissue does not exhibit properties of original tissue, the healing of soft tissue is called repair instead of regeneration. (Bennet N T et al. Am. J. Surg. 1993, 165: 728-737; Bennet N T et al. AM. J. Surg. 1993, 166: 74-81).

However, there are impairments of wound healing that may have numerous causes. Hyperglycaemia impedes wound healing, probably by way of inhibiting the proliferation of endothelial cells and fibroblasts (Goodson W H, J Surg Res 1977 22: 221-227). Increased blood-sugar values also reduce the function of leukocytes, which causes the wound to remain in the inflammatory phase. Furthermore, the degree of severity of the preceding trauma may prompt an unfavourable progression of wound healing (Holzieimer R G, 1966, Zentralbl Chir 121:231).

As opposed to scar tissue, which forms in the process of heating of soft-tissue injuries (repair), the original tissue structure is restored completely after bone fractures or bone transplantations (regeneration). In principle, the regenerative processes in the bone resemble the healing pattern of soft-tissue wound healing: Immediately after the injury, PDGF and TGF-β are, i.a., released by the degranulating thrombocytes, followed by the immigration of macrophages and other inflammatory cells that also secrete PDGF and TGF-β, and in addition secrete fibroblast growth factor (FGF), Interleukin-1 (IL-1), and IL-6. It is assumed that this complex interaction of growth factors with the emerging fibrin scaffold represents the initial step in the process of bone healing, with the surrounding tissues such as bone marrow, periosteum and soft tissue considerably contributing to the regeneration.

In addition to the reorganization of the bone marrow cells into areas of different densities, cell division and differentiation are triggered in the osteoblasts lining the bone and in the preosteoblasts of the cambium. The woven bone newly formed by those processes is referred to as the hard callus. In addition to direct ossification, undifferentiated mesenchymal cells and fibroblasts immigrate into the hematoma from the periosteum and the surrounding soft tissue, respectively. Upon an extensive phase of division, a cartilaginous tissue emerges, i.e. the soft callus, whose cells become hypertrophic, mineralize, and are replaced by woven bone once vessels have sprouted in. The final step of completely restoring the original bone structure consists in the modelling of the woven bone into a lamellar bone by the activity of osteoblasts and osteoblasts. Said process is referred to as indirect ossification, since the cartilage formed has still to be replaced by bone (Barnes et al., JBMR 1999, 11:1805-1815).

The healing of bone fractures does not always progress without problems: infections, systemic diseases (e.g. osteoporosis), metabolic diseases (e.g. diabetes mellitus), genetic defects (e.g. osteogenesis imperfecta) and drug treatments (glucocorticoid therapy) may be causes of delayed regeneration.

Apart from the healing of fractures, the transplantation of autologous bone and of bone substitute materials for lifting procedures or for filling of bone defects is gaining in importance increasingly. Here again, it would be desireable, if the regeneration process could be accelerated and bone quality could be improved.

The precise reasons for a decelerated or missing bone regeneration are unknown. From WO 91/13905, WO 91/04035, WO 91/16009, U.S. Pat. No. 5,165,938 and U.S. Pat. No. 5,178,883, it is known that growth factors released from thrombocytes can be used for wound healing.

The activation of thrombocytes is known, for instance, from WO 86/03122. During activation, growth factors for fibroblasts and muscle cells are released. The product obtained by activation may be processed into an ointment using carrier materials such as microcrystalline collagen,

According to WO 90/07931, said ointment may also be used for supporting the growth of hair.

WO 00/15248 describes a composition containing thrombocytic growth factors as well as fibrin and a further polymer. Said composition can be used for healing and treating damages in tissues characterized by low blood circulation and/or reduced regenerative potential, with, in particular, flexible or hyaline fibrocartilages and fascia tissues belonging to those tissues.

The articular cartilage is an avascular tissue with a limited regenerative potential. None of the currently used methods for renewing the articular cartilages of patients suffering from osteo-arthrosis can be regarded as satisfactory. The state of the art is to expand autologous cartilage cells ex vivo and introduce them into the defect under a periostal lobe (Brittenberg M, NEJM 1994).

The objective of the present invention is to provide a drug composition with superior efficacy in tissue regeneration, in particular bone tissue regeneration.

The drug composition according to the invention is characterized in that

it contains microparticles from blood cells and/or tissues which have been purified by differential centrifugation, filtration or affinity chromatography,

it has been subjected to a procedure for virus inactivation and/or virus depletion,

it has been prepared under sterile conditions, and

it is provided in freeze-dried or deep-frozen state.

Preferred embodiments of the invention are defined in the attached claims.

The invention is based upon the finding that the microparticles released from the eukaryotic cells stimulate, i.e., promote, the proliferation of fibroblasts, osteoblasts, and cartilage cells.

The microparticles may be of homologous origin. The term microparticles covers all cell components that may be separated from an aqueous suspension by the methods described in the literature (e.g., centrifugation at 100 000×g/2 h; Forlow S B, Blood 2000). The separated microparticles may be subjected to a procedure for virus depletion and/or virus inactivation. If desired, the drug composition may be provided with growth factors.

The drug composition according to the invention may be prepared by subjecting thrombocytes to an activating treatment in an aqueous medium in order to cause them to release the regeneration-promoting microparticles, whereupon the aqueous medium containing the released microparticles is centrifuged to sedimentate the coarse cell components. The particulate components of the aqueous supernatant thus obtained are recovered in a second centrifugation step at high rotational speed (e.g. 100 000×g) and are subjected to a procedure for virus depletion and/or virus inactivation. An example of an activating treatment is the contacting of the thrombocytes with thrombin, collagen, Ca²⁺-ionophore A23187 and/or protein C5b-9 of the complement system. The drug composition according to the invention is provided in deep-frozen or freeze-dried state.

The drug composition according to the invention may also be applied repeatedly, whereby the consequently higher concentration of microparticles in the wound area permits a faster formation of granulation tissue. Simultaneously, a provisional extracellular matrix of organic (e.g. fibrin, collagen, polyactons etc.) or inorganic materials (calcium phosphate etc.) may be applied, which serves as a carrier substance for growth factors and as a scaffold for immigrating cells.

The covalent binding of the drug composition according to the invention to the above-mentioned matrices maybe accomplished by transglutaminases.

Furthermore, it is possible to provide metal surfaces with the drug composition according to the invention.

Physical, chemical or physical/chemical combination methods as known in the prior art are suitable for virus depletion and/or virus inactivation.

The sterility of the drug composition according to the invention is achieved either by a sterile recovery of the cell concentrates and aseptic further processing or by sterile filtration.

The recovery of the microparticles, the manufacture of the drug composition according to the invention and its effect on osteoblastic cells is exemplified in greater detail in the following.

Recovery of the Microparticles

A thrombocyte concentrate (2×09 cells) is mixed with an excess amount of thyrode buffer (pH=6.4) and is centrifuged for 10 min. at 1200×g. The supernatant is decanted, the thrombocyte pellet is resuspended in 2 ml of DMEM/F12-ITS and is incubated with 10 μM Ca²⁺-ionophore A23187 (Sigma) for 30 min. at room temperature. By said treatment, microparticles are released from the thrombocytes.

Subsequently, centrifugation at 1200×g is continued for another 10 min., whereby a precipitate and a supernatant are formed. The supernatant (=the thrombocyte supernatant), containing the microparticles released from the thrombocytes, is removed and subjected to fiber centrifugation.

In that maker, the microparticles released by the activation of the thrombocytes are separated by centrifugation for 1 h at 14 000×g, 4° C., and the resulting pellet (=microparticle pellet) is resuspended in 2 ml of DMEM/F12-IST.

In order to obtain the microparticles, also thrombin (Baxter, Austria) or other agents as described above may be used instead of the above-described Ca²⁺-ionophore A23187.

Virus Inactivation of the Thrombocyte Supernatant (Photodynamic Virus Inactivation)

8-methoxypsoralen (dissolved in dimethyl sulfoxide [DMSO]) is added to 50 ml of a microparticle suspension prepared according to the above-mentioned process until a final concentration of 300 μg/ml (final concentration of DMSO 0.3%) is achieved. The suspension is irradiated with ultraviolet light from below and above for six hours at 22-27° C. in an atmosphere of 5% CO₂ and 95% N₂ at a pressure of 2 psi so that the entire light intensity will amount to between 3.5 and 4.8 mW/cm² (Lin L. et al. Blood 1989). In this manner, the microparticle suspension is virus-inactivated.

Once virus inactivation has been completed, the suspension may be deep-frozen or freeze-dried as described below.

Deep freezing: Aliquots of 1 ml of the microparticle suspension are shock-frozen at −80° C. for 30-40 minutes and stored at −80° C. Prior to use, the preparation is thawed at room temperature.

Lyophilization: Aliquots of 1 ml of the microparticle suspension are deep-frozen at −80° C. for at least 24 hours and subsequently are freeze-dried in vacuo between −20° C. and −40° C. for 20 to 24 hours. The freeze-dried supernatants are stored at between −20° C. and −80° C. and are rehydrated with a DMEM/F12-medium prior to use,

Virus Inactivation of a Provisional Extracellular Matrix containing Scaffolds (Chemical Virus Inactivation)

Matrices added to a microparticle suspension prepared according to the above-mentioned process are virus-inactivated by the solvent-detergent-method. For that purpose, 1% (by weight) of tri(n-butyl)phosphate and 1% (by weight) of Triton X-100 are added to a matrix suspension at 30° C., and the mixture is shaken for four hours. 5% (by volume) of soybean oil are added and the solvent-detergent-mixture is removed from the matrix suspension by chromatography using a C18-column (Waters, Millipore) (Horowitz B. et al., Blood 1992, 79-826-831; Piet M P. et al., Transfusion 1990, 30:591-598; Piquet Y. et al., 1992, 63:251-256).

The matrices treated by the above-described chemical method of virus inactivation may subsequently also be subjected to photodynamic virus inactivation.

Cultivation of Human Osteoblasts

Primary human osteoblasts may be obtained from bone fragments of about 1-5 mm³. For that purpose, the bone fragments are washed with phosphate-buffered saline solution (PBS) and are cultured for 2-3 weeks at 37° C., 95% air humidity, and 5% CO₂. DMEM/F12 is used as a culture medium, to which 10% fetal calf serum (FCS), antibiotics and fungicides are added.

The osteoblasts growing out of the bone fragments are removed from the cell culture flasks with trypsin (2.5%), diluted 1:3, and cultured under the same conditions (passage 1). For the purpose of cellular proliferation, the procedure is repeated twice. The media and additives can be purchased from Life Technologies, Grand Island, N.Y., USA.

In order to subsequently stimulate the proliferation of osteoblasts by the microparticles to be obtained from the thrombocytes, the osteoblasts are prepared at a density of 10.000 cells/cm² in microliter plates (Packard, Meriden, Conn., USA) and are precultured for 2-4 days in a complete medium, which, for test purposes, is replaced by a serum-free medium. Said medium is a DMEM/F12-medium to which, instead of FCS, a mixture of 5 mg/ml of insulin/transferrin/selenium (ITS, Boehringer Mannheim, GE) is added.

Cultivation of Human Fibroblasts

Primary human fibroblasts may be obtained from pieces of oral mucosa. For that purpose, the pieces of oral mucosa are washed with PBS and are cultured for 2-3 weeks at 37° C., 95% air humidity, and 5% CO₂. DMEM/F12 was used as cell culture medium, to which 10% FCS, antibiotics and fungicides were added. The fibroblasts growing out of the pieces of oral mucosa (gingiva fibroblasts) were removed from the cell culture flasks with trypsil (2.5%), diluted 1:3, and cultured under the same conditions (passage 1). For the purpose of cellular proliferation, the procedure was repeated twice. The media and additives can be purchased from Life Technologies (Gomstein R A, J Periodontol 1999).

Cultivation of Human Chondrocytes

Primary human chondrocytes may be obtained from pieces of articular cartilages. For that purpose, the cartilage pieces are washed with PBS and are chopped, and the cells are released by digestion with a collagenase B solution (0.4% by weight/by volume; Boehringer Mannheim, Germany). The further steps are described above (F Héraud, Ann Rheum Dis 2000).

Miotic Activity of the Microparticles

The microparticle preparations obtained according to the above-mentioned process were examined for their miotic activity.

In order to determine the biological activity, the preparations were diluted in DMEM/F12-IST at a ratio of 1:5. The dilution thus obtained is referred to as the first dilution (I) and corresponds to a microparticle concentration derived from 2×10⁸ thrombocytes/ml.

From a portion of the first dilution (I), a series of dilutions is established at a ratio of 1:5, where the individual dilutions correspond to the supernatants of 4×10⁷ cells/ml (second dilution II), 8×10⁶ cells/ml (third dilution III), 1.6×10⁶ cells/ml (fourth dilution IV), and 3.2×10⁵ cells/ml (fifth dilution V).

Stimulation of the Proliferation of Osteoblasts

Using the five dilutions I, II, III, IV, and V obtained, the proliferation of osteoblasts is stimulated as follows:

4×100 μl of each dilution is cultured with osteoblasts for 24 h. During the final six hours, 1 μCi [³H] thymidine/spot is added, the incorporation rate of which is teen as a measure for the proliferation of osteoblasts. The absorbed radioactivity is determined by liquid scintillation (Packard). DMEM/F12-ITS serves as a control, where the value obtained is taken to be 100%. FIG. 1 shows the proliferation of osteoblasts achieved with dilutions I-V and the control.

FIG. 1 illustrates a dose-dependent proliferation of osteoblasts. As can be seen, the highest concentration (dilution I) incorporates about 3-7 times more [³H]-thymidine into the DNA than the control without microparticles.

Stimulation of the Proliferation of Fibroblasts

Using the five dilutions I, II, III, IV, and V obtained, the fibroblasts are stimulated as described above:

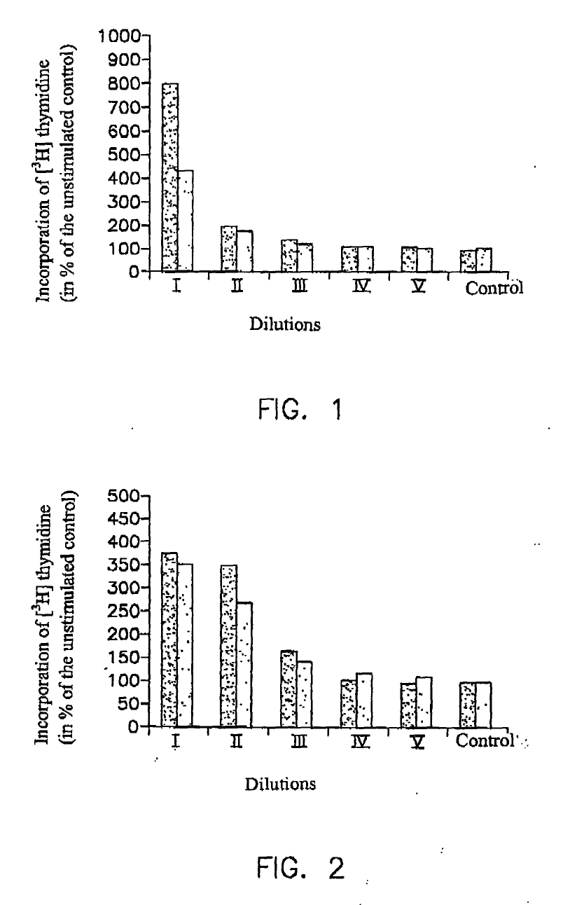

FIG. 2 illustrates a dose-dependent cellular proliferation. As can be seen, the highest concentration (dilution 1) incorporates about 2-3 times more [³H]-thymidine into the DNA than the control without microparticles (black bar: microparticles from donor A, white bar: microparticles from donor B).

Stimulation of the Proliferation of Chondrocytes

Using the five dilutions I, II, III, IV, and V obtained, the chondrocytes are stimulated as described above;

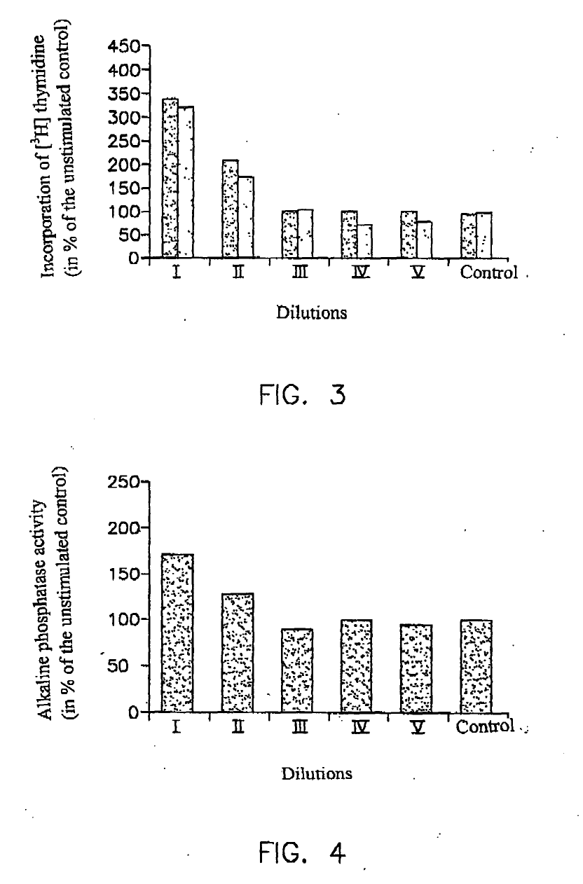

FIG. 3 illustrates a dose-dependent cellular proliferation. As can be seen, the highest concentration (dilution I) incorporates about 2-3 times more [³H]-thymidine into the DNA than the control without microparticles (black bar: microparticles from donor A, white bar: microparticles from donor B).

Stimulation of the Differentiation of Osteoblastic Cells

Using the five dilutions I, II, III, IV, and V obtained, osteoblastic cells are stimulated as follows:

4×100 μl of each dilution are cultured with the osteoblastic cells for four days. The cells are washed with PBS and are lysed in 100 μl of a 0.5% Triton-X100 solution. In each case, 20 μl each of the lysate are used for determining total protein (Gruber R, Cytokine 2000). The measured enzyme activity is correlated with the quantity of total protein to determine the differentiation of osteoblasts. DMEM/F12-ITS is used as a control, where the value obtained is taken to be 100%.

FIG. 4 shows a stimulation of the differentiation of osteoblastic cells using dilution I. The activity of the alkaline phosphates is by about 80% higher than in the control group without microparticles,

Binding of Microparticles to a Provisional Extracellular Matrix containing Scaffolds

A solution of a provisional extracelluar matrix containing scaffolds is added to the sterile, virus-inactivated microparticle suspension prepared according to the above-mentioned process. The scaffolds may be cross-linkable biomaterials (fibrinogen, fibronectin, coagulation factor XIII, collagen), which may have been subjected to one or more procedures for virus inactivation, or organic (e.g. polyactons) or inorganic materials (e.g. calcium phosphates). The components may be used singly or in combination with each other. The mixing ratio of the microparticle suspension with the extracellular matrix should preferably be 1:3. In order to achieve appropriate shelf-life, the mixture is deep-frozen or freeze-dried according to the above-described process.

Instead of applying the virus inactivation to the individual components such as described, it also is possible the apply the virus inactivation to a mixture of the microparticle suspension and the added matrix.

Furthermore, there is a possibility of binding the microparticles to the above-mentioned matrices by a covalent bond by means of transglutaminases.

1. A drug composition to be applied topically for promoting the regeneration of tissue, characterized in that
it contains microparticles from blood cells and/or tissues which have been purified by differential centrifugation, filtration or affinity chromatography,
it has been subjected to a procedure for virus inactivation and/or virus depletion,
it has been prepared under sterile conditions, and
it is provided in freeze-dried or deep-frozen state.

2. A drug composition according to claim 1, characterized in that it contains soluble or insoluble substances promoting wound healing.

3. A drug composition according to claim 1 or claim 2, characterized in that it contains cytokines and/or growth factors.

4. A drug composition according to any of claims 1 to 3, characterized in that it contains a substance which constitutes or may form a provisional extracellular matrix.

5. A drug composition according to any of claims 1 to 4, characterized in that it contains collagen.

6. A drug composition according to any of claims 1 to 5, characterized in that it contains fibrinogen and thrombin for the formation of a fibrin scaffold.

7. A drug composition according to claim 4, characterized in that an organic polymer, in particular a polyacton, is used as the provisional extracellular matrix.

8. A drug composition according to any of claims 1 to 7, characterized in that it contains inorganic compounds.

9. A drug product characterized in that it exhibits:
a drug composition according to any of claims 1 to 8 and
a biocompatible material which is applied together with the drug composition.

10. A drug product according to claim 9, characterized in that the biocompatible material is titanium or an apatite.

11. A process for promoting the regeneration of tissue, in particular the regeneration of bone tissue, characterized in that a drug composition according to any of clams 1 to 8 is applied together with a biocompatible material, in particular titanium or an apatite.

12. The use of an aqueous suspension which contains virus-inactivated microparticles from blood cells and/or tissues for the preparation of a drug composition for accelerating cell growth, in particular the growth of of osteoblasts.
